(19) United States
(12) Patent Application Publication
(10) Pub. No.: US 2005/0003999 A9
(43) Pub. Date: Jan. 6, 2005

(54) ENZYMATIC TREATMENT OF WHEY PROTEINS FOR THE PRODUCTION OF ANTIHYPERTENSIVE PEPTIDES, THE RESULTING PRODUCTS AND TREATMENT OF HYPERTENSION IN MAMMALS

(76) Inventors: Martin E. Davis, Tonka Bay, MN (US); Anand Rao, Savage, MN (US); Sylvie Gauthier, Charny (CA); Yves Pouliot, Charny (CA); Line Gourley, Quebec (CA); Anne-Francoise Allain, Saint-Lambert de Lauzon (CA)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 10/291,860

(22) Filed: Nov. 8, 2002

Related U.S. Application Data
(63) Continuation of application No. PCT/US01/14797, filed on May 8, 2001.
Application No. PCT/US01/14797 is a continuation-in-part of application No. 09/702,068, filed on Oct. 30, 2000.
Application No. PCT/US01/14797 is a continuation-in-part of application No. 09/567,283, filed on May 8, 2000, now Pat. No. 6,630,320.
(63) Continuation-in-part of application No. 09/702,068, filed on Oct. 30, 2000.
Application No. 09/702,068 is a continuation of application No. 09/359,201, filed on Jul. 22, 1999, now abandoned.
(63) Continuation-in-part of application No. 09/567,283, filed on May 8, 2000, now Pat. No. 6,630,320.
(60) Provisional application No. 60/135,080, filed on May 20, 1999.

(51) Int. Cl. A61K 38/17

(52) U.S. Cl. 514/2

(57) ABSTRACT

Enzymatic digests of whey protein concentrates were prepared using animal, bacterial and fungal proteases, and evaluated for antihypertensive activities. ACE-inhibitory activity and antihypertension activity were obtained with a hydrolysate of whey protein isolate prepared with a porcine trypsin.

### BACKGROUND OF THE INVENTION

The invention in one aspect relates to a method for suppressing angiotensin-converting enzyme (ACE), a composition effective for this purpose and a method for preparing the composition, specifically by enzymatic conversion of whey proteins.

In another aspect, the invention relates to a method for reducing hypertension in mammals with specific hydrolysates obtained by the enzymatic conversion of whey proteins.

Hypertension has been reported to be the most important cause of human deaths in industrialized countries. (See, for example, Laragh, J. H., 1979. L'hypertension. Recherche, 105 (10): 1068-1076.) Nearly 30% of the fatalities among adults would result from hypertension or from its renal, coronary or neurological complications. The elucidation of the physiological mechanisms responsible for hypertension has lead the pharmaceutical industry to propose angiotensin converting enzyme (ACE)—inhibitory substances. ACE catalyses the degradation of angiotensin I into angiotensn II, a strong vasoconstrictor.

Peptides found in Brazilian snake venom have been identified as the most effective natural substance for the inhibition of ACE. (See, Ferreira, S. H., Bartelt, D. C., Greene, L. J., 1970. Isolation of bradykinin-potentiating peptides from Bothrops jararaca venom. Biochemistry, 9 (13): 2583-2593.) The inhibitory effect of natural peptides has been related to their binding at the active site of ACE. (See, Maubois, J. L., Léonil, J., Trouvé, R., Bouhallab, S., 1991. Les peptides du lait á activité physiologique III. Peptides du lait á effet cardiovasculaire: activités antithrombotique et antihypertensive. Lait, 71: 249-255.)

A structure-function study of these various bioactive peptides has suggested that they often possess a Pro-Pro, Ala-Pro or Ala-Hyp at their C-teminal sequence. (See, Maruyama, S., Suzuki, H., 1982. A peptide inhibitor of angiotensin I converting enzyme in the tryptic hydrolysate of casein. Agric. Biol. Chem. 46 (5): 1393-1394; and Oshima, G., Shimabukuro, H., Nagasawa, K. 1979. Peptide inhibitors of angiotensin I-converting enzyme in digests of gelatin by bacterial collagenase. Biochim. Biophys. Acta, 566: 128-137.) The occurrence of proline might also contribute to the ACE-inhibitory activity of peptides derived from food proteins. (See, Kohmura, M., Nio, N., Kubo, K., Minoshima, Y., Munekata, E., Ariyoshi, Y. 1989. Inhibition of angiotensin-converting enzyme by synthetic peptides of human β-casein. Agric. Biol Chem., 53 (8): 2107-2114.)

Maruyama and Suzuki [supra] have evidenced such amino acid sequences in peptides from tryptic casein hydrolysates. The authors have shown that the peptide f23-34 from xₛ₁ casein (bovine, variant B), possesses ACE-inhibitory activity estimated by an IC₅₀ value (concentration needed to inhibit 50% ACE activity) of 77 μM. Numerous other studies followed this work and revealed other ACE-inhibitory peptides in casein hydrolysates. In a recent review, Nakano has reported the occurence of 18 distinct milk protein-derived peptide sequences, found in sour milk, and which have been shown to possess ACE-inhibitory activity. (Nakano, T., 1998, Milk derived peptides and hypertension reduction. Int. Dairy J., 8: 375-381.)

However, only a few studies have reported the occurrence of ACE-inhibitory activities among whey proteins hydrolysates. Abubakar, et al., have determined the ACE-inhibitory activity in whey protein hydrolysates using seven different enzymes: trypsin, proteinase-K, actinase-E, thermolysin, papain, pepsin and chymotrypsin. It was shown that the specificity of the enzyme had a pronounced effect on the resulting ACE-inhibitory activity of the hydrolysate, and that the biological activity was originating from the major whey proteins (β-lg, α-la, BSA, Ig) and not from the caseinomacropeptide. (Abubakar, A., Saito, T., Aimar, M. V., Itoh, T. 1996. New derivation of the inhibitory activity against angiotensin converting enzyme (ACE) from sweet cheese whey. Tohoku J. Agric. Res., 47 (1-2): 1-8.) More recent work from Abubakar, et al., has allowed the identification of nine peptide sequences, namely β2-microglobulin (f18-20), β-lactoglobulin (f78-80), serum albumin (f221-222), β-casein (f59-61, f59-64, f62-63, f80-90, f157-158, f205-206), among which β-lactoglobulin (f78-80) showed the strongest antihypertensive activity in spontaneously hypertensive rats. (Abubakar, A., Saito, T., Kitazawa, H., Kawai, Y., Itoh, T., 1998, Structural analysis of new antihypertensive peptides derived from cheese whey protein by proteinase K digestion. J. Daisy Sci., 12: 3131-3138.) Finally, Mullaly et al., have demonstrated that a peptidic fraction, isolated by using RP-HPLC, from a tryptic hydrolysate prepared with bovine β-lactoglobulin had an IC₅₀ value of 159.8 μmol/L, compared to Captopril, a commercial drug commonly used in hypertension treatment, which has an IC₅₀ of 0,006 μmol/L. (Mullally, M. M., Meisel, H., FitzGerald, R. J., 1997. Identification of a novel angiotensin-I-converting enzyme inhibitory peptide corresponding to a tryptic fragment of bovine β-lactoglobulin. FEBS Letters, 402: 99-101.) Mass spectrometry analyses have allowed the identification of peptide f142-148 from β-lactoglobulin as being responsible for the ACE-inhibitory activity in tryptic hydrolysates of β-lactoglobulin. The same peptidic sequence obtained by chemical synthesis showed an IC₅₀ of 42.6 μmol/L.

It would be desirable to provide improvements in the field of suppressing angiotensin-converting enzyme (ACE) and to identify additional materials capable of treating hypertension or its symptoms, especially materials that can be easily employed as part of a simple regimen such as being included in food items.

### SUMMARY OF THE INVENTION

It is an object of the invention to provide improvements in the field of suppressing angiotensin-converting enzyme (ACE).

It is an object of the invention to provide a method for suppressing angiotensin-converting enzyme.

It is another object of the invention to provide a composition effective for suppressing angiotensin-converting enzyme.

It is yet another objective of the invention to provide a method for preparing a composition effective for suppressing angiotensin-converting enzyme, specifically by enzymatic conversion of whey proteins.

It is an object of the invention to provide improvements in the field of treating hypertension in mammals.

It is an object of the invention to provide a method for reducing heart rate and blood pressure associated with hypertension in mammals.

These and other objects are accomplished by one aspect of the invention, by improvements which enable the production of an ACE-suppressing composition by the hydrolysis of whey protein hydrolysate, the recovery of the ACE-suppressing composition, and a regimen for use of the ACE-suppressing composition.

The process for preparing the ACE-suppressing composition comprises: preparing an aqueous solution of whey protein isolate and a proteolytic enzyme; holding said solution under conditions effective to partially hydrolyze said whey protein isolate to provide a hydrolysate having increased ACE-suppressing activity in mammals; and recovering said hydrolysate from said solution. The proteolytic enzyme is inactivated as necessary, preferably by heating. The hydrolysate is preferably dried for use in a regimen which comprises oral administration to a mammal, such as a human or a domestic pet such as a dog or cat, in amounts and at intervals effective to suppress ACE-activity.

Other objects are accomplished by another aspect of the invention by a mammalian treatment regimen entailing orally administration to humans or other mammals effective amounts of a hypertension-reducing composition obtained by: preparing an aqueous solution of whey protein isolate and a proteolytic enzyme; holding said solution under conditions effective to partially hydrolyze said whey protein isolate to provide a hydrolysate having the ability to reduce hypertension in mammals; and recovering said hydrolysate from said solution. The proteolytic enzyme is inactivated as necessary, preferably by heating. The hydrolysate is preferably dried for use in a regimen which comprises oral administration to a mammal, such as a human or a domestic pet such as a dog or cat, in amounts and at intervals effective to reduce hypertension in mammals. Many of the preferred aspects of the invention are described below.

Many of the preferred aspects of the invention are described below.

### BRIEF DESCRIPTION OF THE DRAWINGS

FIGS. 1-4 summarize the results of laboratory testing for antihypersion treatment discussed in detail below.

FIG. 1. Changes in heart rate (A) and mean arterial blood pressure (B) elicited by single oral administration (time 0) of vehicle (0.2 mM PBS pH 7.2; O, n=36 rats) or 601 at the dose of 30 (n=9 rats), 75 (n=8) or 150 mg/kg (n=7) in conscious, unrestrained spontaneously hypertensive rats (SHR). Each point indicates the mean with standard errors shown by vertical lines. *P<0.05 versus vehicle-treated group, ANOVA followed by Fisher's test. HR, heart rate; bpm, beats per minute; MAP, mean arterial blood pressure.

FIG. 2. Changes in heart rate (A) and mean arterial blood pressure (B) elicited by single oral administration (time 0) of vehicle (0.2 mM PBS pH 7.2; O, n=36 rats) or 603K at the dose of 30 (n=10 rats), 75 (n=10) or 150 mg/kg (n=10) in conscious, unrestrained spontaneously hypertensive rats (SHR). Each point indicates the mean with standard errors shown by vertical lines. *P<0.05 versus vehicle-treated group, ANOVA followed by Fisher's test. HR, heart rate; bpm, beats per minute; MAP, mean arterial blood pressure.

FIG. 3. Changes in heart rate (A) and mean arterial blood pressure (B) elicited by single oral administration (time 0) of vehicle (0.2 mM PBS pH 7.2; O, n=36 rats) or 605K at the dose of 30 (n=8 rats), 75 (n=8) or 150 mg/kg (n=8) in conscious, unrestrained spontaneously hypertensive rats (SHR). Each point indicates the mean with standard errors shown by vertical lines. HR, heart rate; bpm, beats per minute; MAP, mean arterial blood pressure.

FIG. 4. Changes in heart rate (A) and mean arterial blood pressure (B) elicited by single oral administration (time 0) of vehicle (0.2 mM PBS pH 7.2; O, n=36 rats) or BiPRO at the dose of 30 (n=8 rats), 75 (n=8) or 150 mg/kg (n=8) in conscious, unrestrained spontaneously hypertensive rats (SHR). Each point indicates the mean with standard errors shown by vertical lines. HR, heart rate; bpm, beats per minute; MAP, mean arterial blood pressure.

### DETAILED DESCRIPTION OF THE INVENTION

Whey protein isolates (WPI) can be obtained from commercial-scale fractionation of cheese whey by various processes, including ion-exchange processing using cationic and/or anionic resins selected for the intended functionality of the isolate. (Pearce, R. J., 1992, Whey protein recovery and whey protein fractionation, Whey and Lactose Processing, J G Zadow, Ed., Elsevier, London, 271-316.) Commercial WPI products issued from ion-exchange processing, such as BiPRO™ (Davisco Foods International, Inc., LeSueur, Minn.), are characterized by a high protein content (>94% w/w), low ash content (<3%), traces (<1%) of fat and lactose. The protein distribution of a typical WPI shows 55-65% β-lactoglobulin, 17-25% α-lactalbumin, 4-7% bovine serum albumin, 7-11% inmmunoglobulins and less than 1% others.

BiPRO™ whey protein isolate is the preferred source of whey protein isolate for use in the invention and is available from Davisco Foods International, Inc., with offices at 11000 W. 78th Street, Suite 210, Eden Prairie, Minn. 55344. The preferred BiPRO™ whey protein isolate has a (PDCAAS) Protein Digestibility Corrected Amino Acid Score of 1.14. The fat and lactose levels are less than 1%. The BiPRO™ whey protein isolate is prepared by ion-exchange technology, and contains about 55-65% (w/w) β-lactoglobulin. Preferaby, the whey protein isolate employed according to the invention will contain at least 55% and preferably at least 60% β-lactoglobulin, with the remaining comprising α-lactalbumin, serum albumin and immunoglobulins in the above ranges. BiPRO™ is essentially undenatured and is fully soluble over the pH range 2.0 to 9.0, and has the following analysis:

| Analysis* | Specification | Typical Range | Test Method |
|---|---|---|---|
| Moisture (%) | 5.0 max. | 4.9 ± 0.1 | Vacuum Oven |
| Protein, dry basis | 95.0 min. | 97.8 ± 0.4 | Combustion |
| (N × 6.38) (%) | | | |
| Fat (%) | 1.0 max. | 0.3 ± 0.1 | Mojonnier |
| Ash (%) | 3.0 max. | 2.0 ± 0.3 | Gravimetric |
| Lactose (%) | 1.0 max. | <0.5 | by difference |
| pH | 6.7-7.5 | 7.0 ± 0.1 | 10% Sol. @ 20° C. |

*All results reported “AS IS” basis except where noted.
Standard Methods for the Examination of Dairy Products, 16th Edition.

As noted, whey protein isolates other than BiPRO™ can be employed and where used preferably have similar analyses to that above, varying by from 0 to 25%, e.g., from 5 to 10%, or less, from the above Typical Range values. A suitable whey protein isolate can be produced having similar properties through a selective ion exchange process that selects the primary functional proteins—beta-lactoglobulin and alpha-lactalbumin—for concentration and spray drying. Such a process is described in U.S. Pat. No. 4,154,675 to Jowett, et al., and U.S. Pat. No. 4,218,490 to Phillips, et al. If properly produced, whey protein fractions having lower protein contents, e.g., as low as 35%, might be employed. In addition, β-lg produced by ion exchange separation can also be employed, but is less preferred than the BiPRO™ whey protein isolate.

On a more detailed analysis of BiPRO™ whey protein isolate, the following is found for each 100 grams of whey protein isolate:

Component

| | |
|---|---|
| Calories | 374 |
| Calories From Total Fat | 3 |
| Total Fat (g) | 0.3 |
| Saturated Fat (g) | 0.2 |
| Cholesterol (mg) | 10 |
| Sodium (mg) | 600 |
| Potassium (mg) | 120 |
| Total Carbohydrates (g) | 0 |
| Dietary Fiber (g) | 0 |
| Sugars (g) | 0 |
| Protein (g) | 93 |
| Vitamin A (IU) | 20 |
| Vitamin C (mg) | 2.0 |
| Calcium (mg) | 120 |
| Iron (mg) | 5 |
| Phosphorus (mg) | 25 |
| Magnesium (mg) | 15 |
| Ash (g) | 2 |
| Moisture (g) | 5 |

And, to provide an amino acid profile of the preferred BiPRO™ whey protein isolate, samples were subjected to drying for 24 hours in a dessicator over phosphorus pentoxide and sodium hydroxide. The dry samples were hydrolyzed in HCl vapor (6N HCl with 1% phenol and 0.5% sodium sulfite) under Argon atmosphere. After 20 hours of hydrolysis at 110 degrees Celsius, the samples were dissolved in 200 μl of Beckman Na—S sample buffer. This acid hydrolysis method destroys tryptophan.

Analyses were conducted on a Beckman 6300 Amino Acid Analyzer. Norleucine was used as an internal standard. The analysis showed the following:

Grams Amino Acid Per

| Amino Acid | 100 g protein | 100 g powder |
|---|---|---|
| Alanine | 7.6 | 7.01 |
| Arginine | 2.0 | 1.84 |
| Aspartate | 10.1 | 9.31 |
| Cysteine/Cystine | 4.3 | 3.96 |
| Glutamine | 14.3 | 13.18 |
| Histidine | 1.6 | 1.48 |
| Isoleucine * | 5.4 | 4.98 |
| Leucine * | 13.7 | 12.63 |
| Lysine * | 9.6 | 8.85 |
| Methionine * | 2.4 | 2.21 |
| Phenylalanine * | 3.1 | 2.86 |
| Proline | 4.5 | 4.14 |
| Serine | 4.90 | 4.52 |
| Threonine * | 5.30 | 4.89 |
| Tyrosine | 2.90 | 2.67 |
| Valine * | 5.60 | 5.16 |
| Totals | 100.10 | 92.29 |

* Essential Amino Acids

Again, when whey protein isolates other than BiPRO™ are employed, they preferably have similar analyses to that above, varying by from 0 to 25%, e.g., from 5-10%, or less, from the above values.

Enzymatic digests of BiPRO™ and of commercial β-lg-rich product were prepared using animal, bacterial and fungal proteases, in order to determine the potential of these commercial substrates for the preparation of peptide mixtures having antihypertensive activities. The objective of the work was to determine the ACE-inhibitory activity of various hydrolysates generated by enzymatic hydrolysis from whey protein isolates obtained by ion-exchange chromatography, in comparison with other commercially-available whey protein hydrolysates.

Materials and Methods for ACE Inhibition

Whey protein hydrolysate WPH 917 (84.5% protein w/w) was obtained from New Zealand Milk Product Inc. (Santa Rosa, USA). Whey protein hydrolysate LE80GF (80.0% w/w) was obtained from DMV International (New-York, USA). Whey protein isolate (BiPRO™) and β-lactoglobulin-rich product were obtained from Davisco Foods International (Le Sueur, Minn., USA). Purified peptidic sequence Ala-Leu-Pro-Met-His-Ile-Arg modelling the peptide f-142-148 from β-lg was chemically-synthetized by the Service de séquence de peptides de l'Est du Québec (Ste-Foy, Qc, Canada). HEPES Sodium salt, Hippuryl-L-Histidyl-L-Leucine, and Angiotensin Converting Enzyme (from rabbit lung) were purchased from Sigma Chemical Co. (St. Louis, USA). All other products used were analytical grade.

Whey proteins (BiPRO™ or β-lg) were solubilized at 20% W/V, adjusted to pH 8.0 or 8.5 by using a mixture of NaOH and KOH 4N and maintained at temperatures between 40° C. and 50° C. corresponding to the optimal temperature of the enzymes used. Table 1 reports the characteristics of the enzymes used for the preparation of the enzymatic hydrolysates for the study. BiPRO™ and β-lg-rich product were utilized for the preparation of 601 and 605, but only Bipro™ was used for 603K. The protein solutions were incubated with the proteases at an enzyme:substrate ratio of 1:800 for AS-601, 1:50 for AS-603K and 1:100 for AS-605K. The enzymatic hydrolysis was performed under pH-stat conditions until a degree of hydrolysis (DH) of 5.5-6.5% for AS-601 and under a combination of pH-stat and osmometry methods until a DH of 11.0-12.5% for AS-603K, and a DH of 19.5-20.5% for AS-605K. The hydrolysis reaction was stopped at the selected DH values by means of heat treatment (75 to 85° C. for 15 s) in a plate heat exchanger to inactivate the enzyme and followed by cooling and storage at 5-10° C. until further processing. The resulting hydrolysates were further spray dried and handled as powdered ingredient. Fractions can be taken based on molecular weight and tested for relative activity, with the most active fractions selected.

A typical analysis for the AS-601K and AS-603K products prepared from BiPRO™ whey protein hydrolysate are given below.

| Analysis* (AS-601K) | Specification | | Typical Range | Test Method |
|---|---|---|---|---|
| Moisture (%) | 5.0 | max. | 4.0 ± 0.5 | Vacuum Oven |
| Total Nitrogen (TN), % | 14.1 | min. | | Combustion |
| Protein dry basis | 90.0 | min. | 91.0 ± 0.5 | Calculated |
| (N × 6.38) (%) | | | | |
| Amino Nitrogen (AN), % | 1.7 | min. | | Formol |
| | | | | Titration |
| AN/TN, % | 12.0 | min. | 12.0-15.0 | Calulated |
| Degree of Hydrolysis, % | 5.0 | min. | 5.5-6.5 | OPA Method |
| Fat (%) | 1.0 | max | <1.0 | Mojonnier |
| Ash (%) | 6.0 | max. | 5.5 ± 0.3 | Standard** |
| Lactose (%) | 1.0 | max. | <1.0 | by difference |
| pH | 8.5 | max. | 8.0 ± 0.2 | 10% Sol. @ |
| | | | | 20° C. |

| Molecular Weight Profile (HPLC) Range (Daltons) | Soluble Peptides*** |
|---|---|
| >5000 | 50-55% |
| 2000-5000 | 15-20% |
| <2000 | 30-35% |

*All results reported “AS IS” basis except where noted.
**Standard Methods for the Examination of Dairy Products, 16th Edition.
***Percent of total soluble peptides in 0.45μ filtrate.

| Analysis* | | | | |
|---|---|---|---|---|
| (AS-603K) | Specification | | Typical Range | Test Method |
| Moisture (%) | 5.0 | max. | 4.0 ± 0.5 | Vacuum Oven |
| Total Nitrogen | 14.1 | min. | | Combustion |
| (TN), % | | | | |
| Protein, dry basis | 94.0 | min. | 95.0 ± 0.5 | Calculated |
| (N × 6.38) % | | | | |
| Amino Nitrogen | 2.2 | min. | 2.4 ± 0.2 | Formol |
| (AN), % | | | | Titration |
| AN/TN, % | 15.8 | min. | 16.3 ± 0.5 | Calculated |
| Degree of | 7.7 | min. | 8.7 ± 1.0 | OPA Method |
| Hydrolysis, % | | | | |
| Fat (%) | 1.0 | max. | <1.0 | Mojonnier |
| Ash (%) | 5.0 | max. | 3.5 ± 0.3 | Gravimetric |
| Lactose (%) | 1.0 | max. | <1.0 | by difference |
| pH | 7.5 | max. | 7.0 ± 0.2 | 10% Sol. @ |
| | | | | 20° C. |
| Scorched | 15 mg/25 g | max. | 7.5 mg | ADPI |
| Particles | | | | |

| | | |
|---|---|---|
| Molecular Weight Profile | | |
| (HPLC) | Range (Daltons) | Peptides |

| | |
|---|---|
| >10,000 | 30-35% |
| 5000-10,000 | 10-15% |
| 2000-5000 | 25-30% |
| <2000 | 25-30% |

TABLE 1
Characteristics of the enzyme sources used for the preparation of
hydrolysates

| Enzyme | | Optimal | Temp. | |
|---|---|---|---|---|
| (Name, Supplier) | Source | pH | (° C.) | Hydrolysate |
| Trypsin VI | Porcine | 8.0 | 37 | AS-601 (BiPRO ™) |
| Trypsin Activity | | | | |
| 2,400 U/mg | | | | |
| minimum | | | | |
| Chymotrypsin | | | | |
| activity 350 U/mg | | | | |
| minimum | | | | |
| Canadian | | | | AS601 (β-lg) |
| Innnovatech Inc | | | | |
| Abbotsford, BC, | | | | |
| Canada | | | | |
| Protease P | Fungal | 7.5 | 45 | AS-605K (BiPRO ™) |
| Amano-6 | | | | |
| Proteinase activ- | | | | |
| ity 60,000 units/g | | | | |
| minimum | | | | |
| Amano, Enzyme | | | | AS-605K (β-lg) |
| Co. Ltd | | | | |
| Rochester, IL, | | | | |
| USA | | | | |
| Multifect | Bacterial | 8.5 | 50 | AS-603K (BiPRO ™) |
| Activity 3,000 | | | | |
| GSU/ml | | | | |
| minimum (Note: | | | | |
| GSU is Genencor | | | | |
| Subtlisin Units— | | | | |
| developed intern- | | | | |
| ally by | | | | |
| Genencor) | | | | |
| Genencor | | | | |
| International | | | | |
| Rochester, NY, | | | | |
| USA | | | | |

Determination of ACE-Inhibitory Activity

The ACE-inhibitory activity was measured in vitro by a spectrophotometric assay according to the method of Cushman and Cheung. (Cushman, D. N., Cheung, H. S. 1971 Spectrophotometric assay and properties of the angiotensin converting enzyme of rabbit lung. Biochemical Pharmacology, 20: 1637-1648.) According to this method, hippuric acid is liberated from hippuryl-L-histidyl-L-leucine (HHL) by the enzymatic reaction of ACE. After extraction by ethyl acetate and removal of ethyl acetate by heat evaporation, hippuric acid is dissolved in deionized water.

Table 2 summarizes the experimental conditions used for the assay. Absorbance of the hippuric acid solution at 228 nm was determined by spectrophotometry.

TABLE 2
Assay conditions for the measurement of ACE-inhibitory activity

| Reagent volumes (μl) | Sample | Control | Blank |
|---|---|---|---|
| Substrate solution¹ | 200 | 200 | 200 |
| Sample solution² | 50 | — | — |
| HEPES-HCl Buffer³ | — | 50 | 50 |
| Deionized water | 20 | 20 | 20 |

mixed using vortex and equilibrated to 37° C.

| | | | |
|---|---|---|---|
| Stopping solution⁴ | — | — | 300 |
| ACE solution⁵ | 30 | 30 | 30 |

mixed using vortex and incubated at 37° C. for 20 min

| | | | |
|---|---|---|---|
| Stopping solution | 300 | 300 | — |
| Total volume (μl) | 600 | 600 | 600 |

¹HHL was dissolved in HEPES-HCl Buffer to obtain a final concentration of 0.3% (w/v).
²Samples were diluted at the appropriate concentration with HEPES-HCl Buffer.
³HEPES Sodium Salt (50 mM) with 300 mM NaCl, pH adjusted at 8.3 with 1M HCl
⁴1M HCl.
⁵ACE from rabbit lung dissolved in HEPES-HCl at a final concentration of 0.33 unit ml⁻¹.

Inhibitory activity was calculated according to the following equation:
Inhibitory activity (%)=[(A_{control}−Aₛₐₘₚₗₑ)/(A_{control}−A_{blank})]×100 [1]
where A represents absorbance. A plot of the inhibitory activity (%) versus log₁₀ of sample concentration (mg powder ml⁻¹) was generated using 6 different concentrations of samples for BiPRO™, AS-601 (BiPRO™β-lg), commercial hydrolysates (WPH 917, LE80GF) and of synthetic peptide (f142-148) from β-lg. Each concentration was tested in triplicate and the mean value was plotted in the curves. The IC₅₀ value (expressed in terms of mg powder ml⁻¹, defined as the concentration of inhibitor which gives 50% inhibition of ACE activity, was calculated using the linear regression equations of the curves.

The linear regression equations corresponding to all the hydrolysates under study are reported in Table 3. The data show that non-hydrolyzed BiPRO™ has very low ACE-inhibitory activity (IC₅₀ 380 mg powder. ml⁻¹) in comparison with all other products under study. The highest ACE-inhibitory activity was obtained with synthetic peptide f142-148 of β-lg for which 50% of inhibition was already obtained at the lowest concentration (IC₅₀ 0.04 mg powder.ml⁻¹). The ACE-inhibitory effectiveness of the hydrolysates under study followed the order: AS-603K (BiPRO™)>AS-605K (BiPRO™)>AS-601 (BiPRO™)>AS-605K (β-lg)>AS-601 (β-lg)>LE80GF>WPH 917.

Table 3, as follows presents the linear regression equations (y=m lnX+b) of the ACE-inhibitory activity curves obtained with synthetic peptide β-lg (f142-148), in comparison with the hydrolysates under study.

TABLE 3

| | Slope | Y-intercept | Reg. Coeff. |
|---|---|---|---|
| Sample | m | b | R² |
| β-lg f142-148 | 10.35 | 84.4 | .83 |
| AS-601 (BiPRO ™) | 15.39 | 62.4 | .99 |
| AS-605 (BiPRO ™) | 14.76 | 63.0 | .99 |
| AS-603K (BiPRO ™) | 14.33 | 67.9 | .96 |
| AS-601 (β-lg) | 17.69 | 51.9 | .98 |
| AS-605 (β-lg) | 16.98 | 60.7 | .98 |
| WPH 917 (NZMP) | 19.40 | 44.8 | .96 |
| LE80GF (DMV) | 18.01 | 50.7 | .97 |

The values of IC₅₀ calculated for all the samples under study are listed in Table 4. The samples of BiPRO™ showed lower ACE-inhibitory activity with an IC₅₀ of 376 mg powder.ml⁻¹), whereas synthetic peptide f142-148 of β-lg showed the lowest value (0.04 mg powder.ml⁻¹). The hydrolysates derived from BiPRO™ or β-lg-rich products all gave lower IC₅₀ values (0.29 to 0.90 mg powder.ml⁻¹) than the other commercial hydrolysates (0.96 and 1.30 mg powder.ml⁻¹). Also the hydrolysates derived from BiPRO™ led to lower IC₅₀ values than those derived from the β-lg-rich product (ex. AS-601_{BiPRO™}=0.45 vs AS-601_{β-lg}=0.90 powder.ml⁻¹).

TABLE 4
Values of IC₅₀ for various whey protein products

| | | IC₅₀ |
|---|---|---|
| Sample | Description | (mg powder ml⁻¹) |
| BiPRO ™ | whey protein isolate | 376.7 |
| AS-601 (BiPRO ™) | hydrolyzed BiPRO ™ | 0.45 |
| AS-601 (β-lg) | hydrolyzed β-lg | 0.90 |
| AS-605 (BiPRO ™) | hydrolyzed BiPRO ™, Kosher | 0.42 |
| | certified | |
| AS-605 (β-lg) | hydrolyzed β-lg, Kosher | 0.53 |
| | certified | |
| AS-603K (BiPRO ™) | Hydrolyzed BiPRO ™, | 0.29 |
| | Kosher certified | |
| WPH 917 (NZMP) | whey protein hydrolysate | 1.30 |
| LE80GF (DMV) | whey protein hydrolysate | 0.96 |
| β-lg f142-148 | synthetic peptide | 0.04 |

Discussion

Our observations show that the enzymatic hydrolysis of whey proteins issued from ion-exchange chromatography, such as BiPRO™ whey protein isolate generates hydrolysates having superior ACE-inhibitory activities, compared to commercial hydrolysates prepared with other sources of whey proteins. Moreover, it appears that BiPRO™ whey protein isolate is a better substrate than β-lg-rich product for the preparation of hydrolysates with antihypertensive activity, as seen by the lower IC₅₀ values obtained (Table 4) for AS-601, AS-603K and 605K prepared from Bipro™ whey protein isolate. This observation was unexpected since it was hypothesized that a β-lg-rich product would offer a higher potential for producing ACE-Inhibiting peptides, especially the fragment β-lg 142-148 liberated by trypsin (Mullally, M. M., Meisel, H., FitzGerald, R. J., 1997. Identification of a novel angiotensin-I-converting enzyme inhibitory peptide corresponding to a tryptic fragment of bovine β-lactoglobulin. FEBS Letters, 402: 99-101). In fact, the hydrolysates having the lowest IC₅₀ (0.29 mg powder ml⁻¹) were obtained by hydrolysis with bacterial protease (AS-603K) and not with trypsin (AS-601). Altogether, our results suggest the occurrence of a synergistic effect when BiPRO™ whey protein isolate is used as the substrate, but the explanation for this phenomenon is not clear.

BiPRO™ whey protein isolate and others similarly prepared are preferred for a composition with regard to principal protein composition (β-lg, α-la, etc.), and content of minor proteins (lactoferrin, lactoperoxydase, immunoglobulins) or peptidic fragments (caseinomacropeptides, proteoses peptones, etc.) which may be precursors of the production of peptides with very strong ACE-inhibition activity during enzymatic hydrolysis. Some of these minor proteins may be at a lower concentration in the β-lg-rich product, as a result of the different fractionation conditions.

Also, it must be emphasized that the enzymes used for the preparation of enzymatic hydrolysates from BiPRO™ whey protein isolate (601, 605K and 603K) respectively Trypsin VI, P Amano 6 and Multifect have very different specificities. Trypsin is known to cleave only the peptidic bonds in the vicinity of Arg and Lys, whereas the two other enzymes have a much broader specificity and will lead to a greater number of shorter peptides. The recent work from Abubakar, et al., supra, suggests that short molecules such as tri- and even di-peptides can have an antihypertensive effect. It is clear that the fragments 142-148 and 78-80 of β-lg are not the only ACE-inhibitors responsible for the antihypertensive properties of all whey protein hydrolysates.

The synergistic effect on ACE-inhibitory potential of whey protein hydrolysates when BiPRO™ whey protein isolate is used may also be originating from its low mineral content, especially with regards to divalent cations such as calcium (15-20 meq/kg) or magnesium (<1 meq/kg). These physicochemical conditions may prevent the occurrence of peptide-peptide interactions and therefore preserve the high ACE-inhibitory potential of the hydrolysate. This hypothesis will be further investigated by comparing the mineral composition of BiPRO™ whey protein isolate with that of β-lg-rich product which showed a lower ACE-inhibitory potential.

This study has shown that the enzymatic hydrolysis of BiPRO™ leads to a synergistic effect in terms of ACE-inhibitory potential of the hydrolysates obtained. It appears that the synergy could result from unique compositional characteristics brought by the ion-exchange process in relation with the presence of minor proteins or fragments.

Materials and Methods for Treatment of Hypetension in Mammals

Enzymatic digests of BiPRO™ whey protein isolate were prepared using animal, bacterial and fungal proteases, in order to determine the potential of these commercial substrates for the preparation of peptide mixtures having antihypertensive activities. The main objective of the present study was to investigate the antihypertensive effect of some specific whey protein hydrolysates. The following summarizes experiments that have been carried out in conscious spontaneously hypertensive rats (SHR) to characterize the antihypertensive effect and establish the dose response curve for each hydrolysate. The SHR are considered as a genetic model of essential hypertension and are currently used to understand the development and establishment of hypertension and to determine the blood pressure lowering effect of newly synthesized antihypertensive drugs. Moreover, in a previous study carried out in SHR, we found that the intravenous injection of an angiotensin I-coverting enzyme (ACE) inhibitor, Captopril, caused a marked reduction in blood pressure accompanied by significant vasodilations in renal, superior mesenteric and hindquarter vascular beds (unpublished data). Therefore, the SHR appears to be very reactive to ACE inhibition and then should provide us with very useful information in determining if specific whey protein hydrolysates exhibit a significant antihypertensive property.

Three whey protein hydrolysates (namely, 601, 603K and 605K) and controls phosphate buffered saline (PBS) and untreated BiPRO whey protein isolate, have been examined. The effects of each whey protein on mean and phasic arterial blood pressure and heart rate have been investigated in conscious unrestrained male SHR. In the present study, the animals were chronically instrumented with one intravascular catheter implanted in the left femoral artery to permit a continuous recording of blood pressure and heart rate. This technique is very well developed in our laboratory and is routinely performed (Bachelard, H. and G. Drolet, Cardiovascular responses to paraventricular (PVN) injections of opioid agonists in conscious rats, Abstract Society for Neuroscience. 18: 1175,1992; Bachelard, H. and M. Pitre, Regional haemodynamic effects of μ-, δ-, κ-opioid agonists microinjected into the hypothalamic paraventricular nuclei of conscious, unrestrained rats, Br. J. Pharmacol, 115: 613-621, 1995; Pitre, M., H. Gaudreault, M. Santure, A. Nadeau, and H. Bachelard. Isradipine and insulin sensitivity in hypertensive rats, Am. J. Physiol, 39: E1038-E1048, 1999; Pitre, M., A. Nateau and H. Bachelard, Insulin sensitivity and hemodynamic responses to insulin in Wistar-Kyoto and spontaneously hypertensive rats, Am. J. Physiol. 271: E658-E668, 1996). This experimental approach constitutes a powerful tool to investigate the hemodynamic responses to whey proteins in conscious rats. The non-invasive technique currently used by others to measure systolic blood pressure, the tail cuff sphygmomanometer, is stressful for the animal, (the rat has to be immobilized in a restrainer during measurement), and is subject to a large variability. Therefore, considering the inherent variability in blood pressure measurements in conscious animals, and that the SHR rats are known to be very reactive to stressful stimuli, we believed that there is some incertitude in measurements made with the tail cuff technique, particularly in establishing a steady baseline measurement. In contrast, by directly and continuously recording blood pressure, it is easier to take into account this variability and make more reliable blood pressure measurements. Moreover, our rats were well adapted to the system for a few days prior to the experiments, and direct measurement of blood pressure was made without causing any pain or stress to the animal. Therefore, the direct and continuous recording of blood pressure and heart rate in conscious, unrestrained animals represent a clear advantage over the other non-invasive technique.

Whey protein isolate (BiPRO™) was obtained from Davisco Foods International (Le Sueur, Minn., USA). HEPES Sodium salt, Hippuryl-L-Histidyl-L-Leucine, and Angiotensin Converting Enzyme (from rabbit lung) were purchased from Sigma Chemical Co. (St. Louis, USA). All other products used were analytical grade.

Whey proteins (BiPRO™) were solubilized at 20% W/V, adjusted to pH 8.0 or 8.5 by using a mixture of NaOH and KOH 4N and maintained at temperatures between 40° C. and 50° C. corresponding to the optimal temperature of the enzymes used. Table 1 reports the characteristics of the enzymes used for the preparation of the enzymatic hydrolysates for the study. BiPRO™ whey protein isolate was utilized for the preparation of 601, 603K and 605K. The protein solutions were incubated with the proteases at an enzyme:substrate ratio of 1:800 for 601, 1:50 for 603K and 1:100 for 605K. The enzymatic hydrolysis was performed under pH-stat conditions until a degree of hydrolysis (DH) of 4.5-6.5% for 601 and under a combination of pH-stat and osmometry methods until a DH of 7.0-10.0% for 603K, and a DH of 13.0-17.0% for 605K. The hydrolysis reaction was stopped at the selected DH values by means of heat treatment (75 to 85° C. for 15 s) in a plate heat exchanger to inactivate the enzyme and followed by cooling and storage at 5-10° C. until further processing. The resulting hydrolysates were further spray dried and handled as powdered ingredient. Fractions can be taken based on molecular weight and tested for relative activity, with the most active fractions selected.

A typical analysis for the 601K and 603K products prepared from BiPRO™ whey protein hydrolysate are given below.

| Analysis* (601) | Specification | | Typical Range | Test Method |
|---|---|---|---|---|
| Moisture (%) | 5.0 | max. | 4.0 ± 0.5 | Vacuum Oven |
| Total Nitrogen (TN), % | 14.1 | min. | | Combustion |
| Protein dry basis | 90.0 | min. | 91.0 ± 0.5 | Calculated |
| (N × 6.38) (%) | | | | |
| Amino Nitrogen (AN), % | 1.7 | min. | | Formol |
| | | | | Titration |
| AN/TN | 12.0 | min. | 12.0-15.0 | Calulated |
| Degree of Hydrolysis, % | 5.0 | min. | 5.5-6.5 | OPA Method |
| Fat (%) | 1.0 | max | <1.0 | Mojonnier |
| Ash (%) | 6.0 | max. | 5.5 ± 0.3 | Standard** |
| Lactose (%) | 1.0 | max. | <1.0 | by difference |
| pH | 8.5 | max. | 8.0 ± 0.2 | 10% Sol. @ |
| | | | | 20° C. |

| Molecular Weight Profile (HPLC) Range (Daltons) | Soluble Peptides*** |
|---|---|
| >10,000 | 50-60% |
| 5,000-10,000 | 10-20% |
| 2000-5000 | 10-20% |
| <2000 | 10-20% |

*All results reported “AS IS” basis except where noted.
**Standard Methods for the Examination of Dairy Products, 16th Edition.
***Percent of total soluble peptides in 0.45μ filtrate.

| Analysis* (603K) | Specification | | Typical Range | Test Method |
|---|---|---|---|---|
| Moisture (%) | 5.0 | max. | 4.0 ± 0.5 | Vacuum Oven |
| Total Nitrogen (TN), % | 14.1 | min. | | Combustion |
| Protein, dry basis | 94.0 | min. | 95.0 ± 0.5 | Calculated |
| (N × 6.38) % | | | | |
| Amino Nitrogen (AN), % | 2.2 | min. | 2.4 ± 0.2 | Formol |
| | | | | Titration |
| AN/TN, % | 15.8 | min. | 16.3 ± 0.5 | Calculated |
| Degree of Hydrolysis, % | 7.0 | min. | 7-10 | OPA Method |
| Fat (%) | 1.0 | max. | <1.0 | Mojonnier |
| Ash (%) | 5.0 | max. | 3.5 ± 0.3 | Gravimetric |
| Lactose (%) | 1.0 | max. | <1.0 | by difference |
| pH | 7.5 | max. | 7.0 ± 0.2 | 10% Sol. @ |
| | | | | 20° C. |

| Scorched Particles | 15 mg/25 g | 7.5 mg | ADPI |
|---|---|---|---|
| | max. | | |

| | | |
|---|---|---|
| Molecular Weight Profile | | |
| (HPLC) | Range (Daltons) | Peptides |
| | >10,000 | 25-35% |
| | 5000-10,000 | 10-20% |
| | 2000-5000 | 25-35% |
| | <2000 | 20-30% |

The above hydrolysates are characterized by a degree of hydrolysis of from 4.5% to 10.0%. This is determined by OPA Methodology.

TABLE 1
Characteristics of the enzyme sources used for
the preparation of hydrolysates

| Enzyme | | Optimal | Temp. | |
|---|---|---|---|---|
| (Name, Supplier) | Source | pH | ° C. | Hydrolysate |
| Trypsin VI | Porcine | 8.0 | 37 | 601 |
| Trypsin Activity | | | | |
| 2,400 U/mg | | | | |
| minimum | | | | |
| Chymotrypsin activity | | | | |
| 350 U/mg | | | | |
| minimum | | | | |
| Canadian Innovatech Inc | | | | |
| Abbotsford, BC, Canada | | | | |
| Protease P Amano-6 | Fungal | 7.5 | 45 | 605K |
| Proteinase activity | | | | |
| 60,000 units/g | | | | |
| minimum | | | | |
| Amano, Enzyme Co. Ltd | | | | |
| Lombard, IL, USA | | | | |
| Multifect | Bacterial | 8.5 | 50 | 603K |
| Activity 3,000 GSU/ml | | | | |
| minimum (Note: GSU is | | | | |
| Genencor Subtlisin Units- | | | | |
| developed internally by | | | | |
| Genencor) | | | | |
| Genencor International | | | | |
| Rochester, NY, USA | | | | |

Determination of Antihypertension Activity—Methods

All surgical and experimental procedures followed institutional animal care guidelines. Male SHR rats (aged 20 to 24 weeks and weighing 300-350 g) were purchased from Charles River Canada. The rats were anesthetized with a mixture of ketamine-xylazine (100 and 10 mg kg⁻¹), respectively, i.p., supplemented as required) and one catheter was implanted in the distal abdominal aorta via the left femoral artery (for measurement of blood pressure and heart rate). Then, the catheter was tunneled subcutaneously to emerge at the back of the neck and was passed through a flexible, protecting spring attached to a custom-made harness worn by the rat. The rats were given s.c. injections of Ampicillin (150 mg kg⁻¹) and Anafen (5 mg kg⁻¹) and returned to their home cages. Experiments began at least 4 days after this surgical step.

Throughout the experiments, three variables were recorded (heart rate and phasic and mean arterial blood pressure) using a Biopac Data Acquisition and Analysis system (Model MP 100, AcqKowledge software version 3.1). At selected time points heart rate, phasic and mean arterial blood pressure were measured, to represent the full profile of the effects of the hydrolysates or BiPRO whey protein isolate, and related to the pre-treatment baseline value. The rats were conscious, unrestrained and were allowed free access to water and food for the duration of the experiment.

Experimental Protocol

The cardiovascular changes elicited by oral administration of BiPRO whey protein isolate or specific whey protein hydrolysates (601, 603K, 605K) have been evaluated in conscious, unrestrained SHR. The rats were used on four consecutive days, during which they received increasing doses of only one specific hydrolysate (601, 603K, 605K) or the BiPRO by gastric intubation. The lyophilized whey protein hydrolysate powder was dissolved in 0.2 mM PBS (pH 7.2) (the same vehicle, 0.2 mM PBS, was used for control administrations) and was given in a volume of 0.5 ml. All solutions were freshly prepared. Thus, on a typical day, soon after basal measurements of blood pressure and heart rate, the rats were given the vehicle (control PBS on day 1) or an isolated dose of a specific hydrolysate (30 mg/kg on day 2, 75 mg/kg on day 3 and 150 mg/kg on day 4) or BiPRO whey protein isolate (30 mg/kg on day 2, 75 mg/kg on day 3 and 150 mg/kg on day 4). The blood pressure and heart rate effects of a single oral administration of PBS, BiPRO whey protein isolate or the whey protein hydrolysate was evaluated over a period of 7 hours.

Results

Resting values for cardiovascular variables measured every morning before any intervention during four consecutive days are shown in Table 2. As we can see, there was no significant difference between the basal mean arterial blood pressure and heart rate values measured on days 1, 2, 3 and 4.

TABLE 2
Baseline values of heart rate (HR) and mean arterial blood pressure
(MAP) in conscious, unrestrained SHR rats.

| Treatment | Weight, g | Age, Week | Day | HR (bpm) | MAP (mmHg) |
|---|---|---|---|---|---|
| Vehicle(PBS) | 322 ± 4 | 24.1 ± 0.2 | 1 | 311 ± 6 | 173 ± 3 |
| N = 36 | | | | | |
| #601 (30 mg/kg⁻¹) | 321 ± 9 | 23.2 ± 0.5 | 2 | 308 ± 8 | 174 ± 6 |
| N = 9 | | | | | |
| #601 (75 mg/kg⁻¹) | 317 ± 9 | 23.4 ± 0.6 | 3 | 320 ± 13 | 179 ± 4 |
| N = 8 | | | | | |
| #601 (150 mg/kg⁻¹) | 313 ± 10 | 23.0 ± 0.5 | 4 | 305 ± 19 | 175 ± 9 |
| N = 7 | | | | | |
| #603K (30 mg/kg⁻¹) | 315 ± 7 | 23.7 ± 0.2 | 2 | 301 ± 10 | 168 ± 7 |
| N = 10 | | | | | |
| #603K (75 mg/kg⁻¹) | 315 ± 7 | 23.7 ± 0.2 | 3 | 304 ± 9 | 167 ± 5 |
| N = 10 | | | | | |
| #603K (150 mg/kg⁻¹) | 315 ± 7 | 23.7 ± 0.2 | 4 | 292 ± 11 | 164 ± 5 |
| N = 10 | | | | | |
| #605K (30 mg/kg⁻¹) | 321 ± 6 | 24.0 ± 0.0 | 2 | 290 ± 9 | 170 ± 5 |
| N = 8 | | | | | |
| #605K (75 mg/kg⁻¹) | 321 ± 6 | 24.0 ± 0.0 | 3 | 296 ± 14 | 176 ± 9 |
| N = 8 | | | | | |
| #605K (150 mg/kg⁻¹) | 321 ± 6 | 24.0 ± 0.0 | 4 | 299 ± 13 | 163 ± 6 |
| N = 8 | | | | | |
| BiPRO (30 mg/kg⁻¹) | 328 ± 5 | 25.5 ± 0.2 | 2 | 299 ± 9 | 182 ± 5 |
| N = 8 | | | | | |
| BiPRO (75 mg/kg⁻¹) | 328 ± 5 | 25.5 ± 0.2 | 3 | 300 ± 11 | 179 ± 6 |
| N = 8 | | | | | |
| BiPRO (150 mg/kg⁻¹) | 328 ± 5 | 25.5 ± 0.2 | 4 | 290 ± 6 | 168 ± 7 |
| N = 8 | | | | | |

Values are means±SEM; n is the number of rats. (Groups represent those used to assess blood pressure and heart rate effects of single oral administration of vehicle (PBS), 601, 603K, 605K or BiPRO whey protein isolate in conscious, unrestrained SHR. MAP, mean arterial blood pressure; HR, heart rate; bpm, beats per minute.

Cardiovascular Responses to Hydrolysate 601

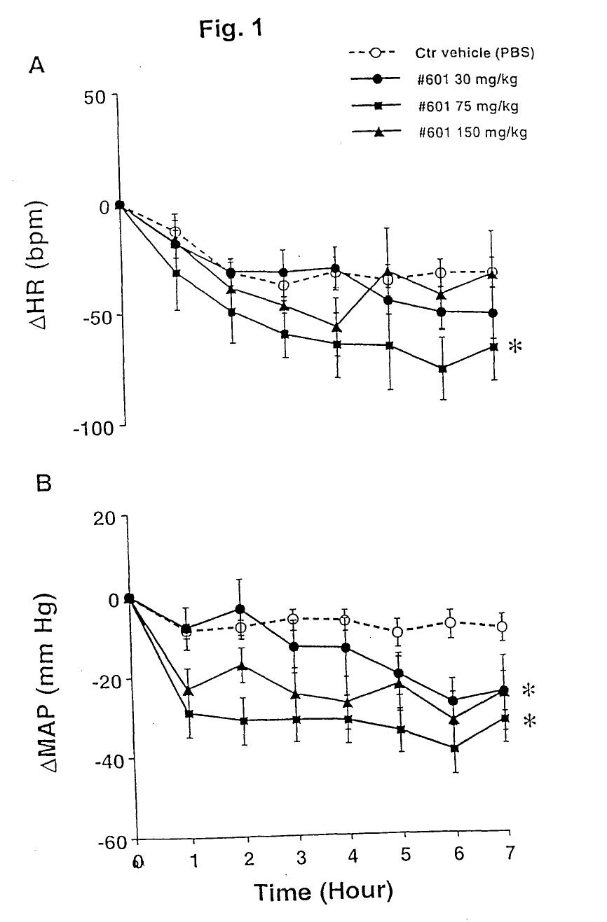

FIG. 1 shows the changes in heart rate and mean arterial blood pressure after oral administration of 0.5 ml of 0.2 mM PBS (n=36) or hydrolysate 601 at doses of 30 (n=9), 75 (n=8) or 150 mg/kg (n=7) in conscious, unrestrained spontaneously hypertensive rats (SHR). Each point indicates the mean with standard errors shown by vertical lines. *P<0.05 versus vehicle-treated group, ANOVA followed by Fisher's test. HR, heart rate; bpm, beats per minute; MAP, mean arterial blood pressure. Except for the 75 mg/kg dose, which elicited a significant (at 4,6 and 7 h) decrease in heart rate (maximum of −77±14 bpm at 6 h), we found no significant difference between the heart rate responses elicited by intra-gastric administration of PBS and hydrolysate 601 at the doses of 30 and 150 mg/kg. However, we observed a significant reduction in mean arterial blood pressure following intra-gastric administration of the different doses of hydrolysate 601 when compared to control administration of PBS.

Thus, at the dose of 30 mg/kg we observed a significant hypotensive effect (−27±6 mm Hg) 6 hours after administration of the hydrolysate. At the dose of 75 mg/kg of hydrolysate we observed a marked and long-lasting hypotensive effect (significant at 1-7 h) when compared with the effects of control administration of PBS. The maximum decrease in mean arterial blood pressure (−39±6 mm Hg) was achieved 6 h after the administration. A similar hypotensive effect was also observed following the intra-gastric administration of 150 mg/kg of the hydrolysate (significant at 1 and 3-7 h). The maximum decrease in mean arterial blood pressure (−32±7 mm Hg) was achieved 6 h after the administration.

Cardiovascular Responses to BiPRO and Hydrolysate 603K, 605K

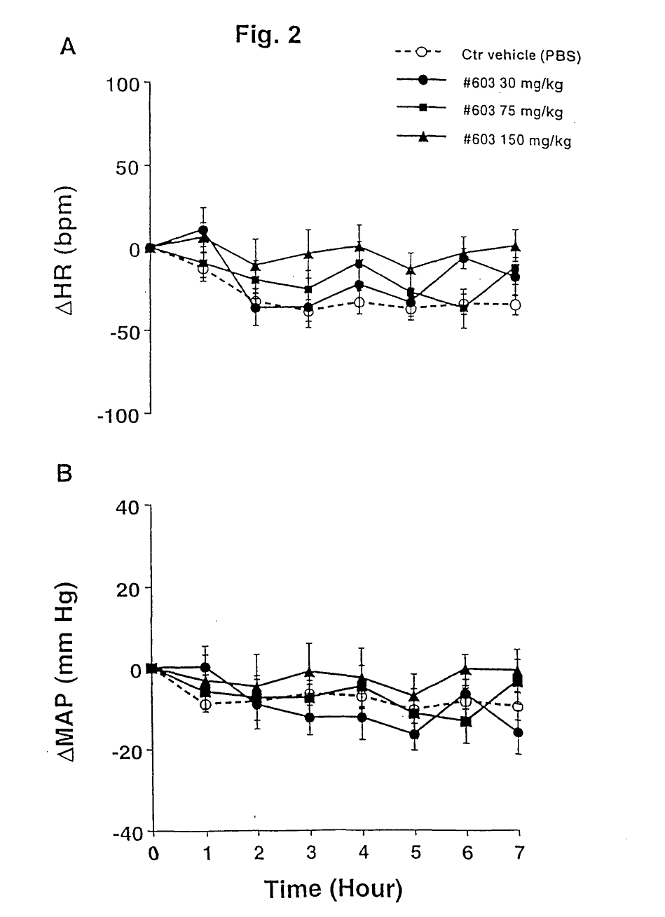

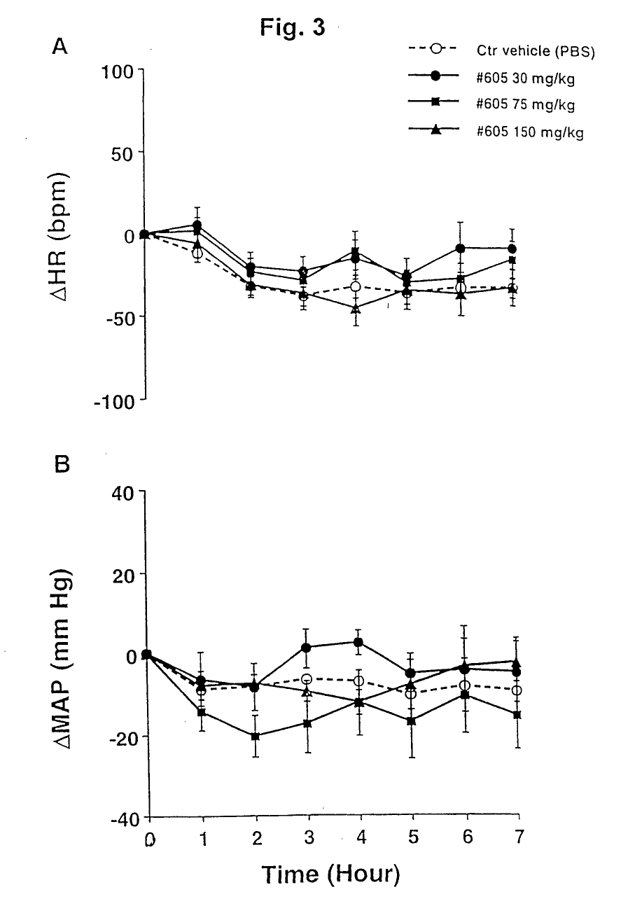

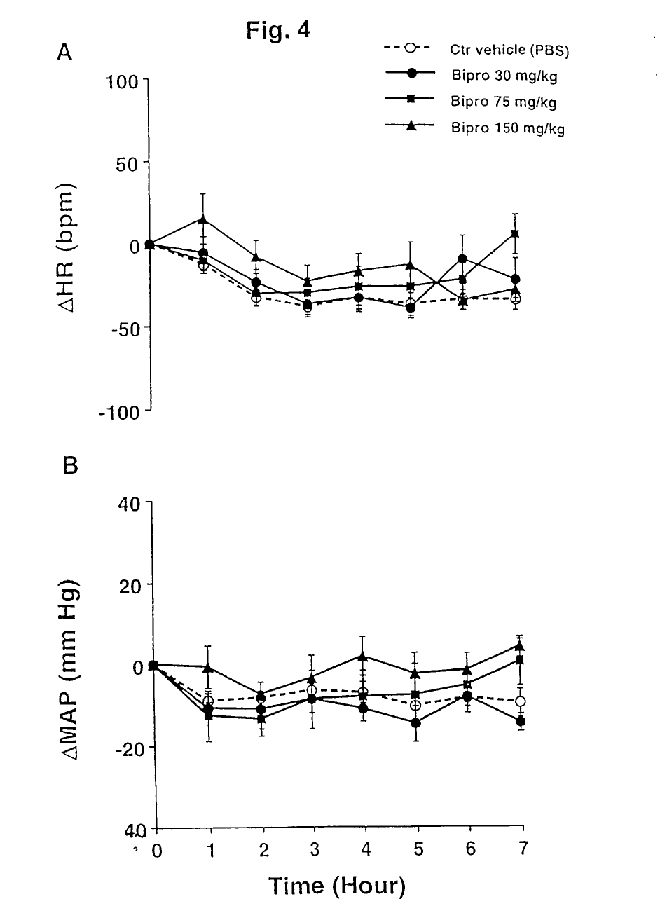

FIGS. 2-4 show the changes in heart rate and mean arterial blood pressure after oral administration of 0.5 ml of 0.2 mM PBS (n=36) or hydrolysate 603K (FIGS. 2) or 605K (FIG. 3) or BiPRO whey protein isolate (FIG. 4) at doses of 30, 75, or 150 mg/kg. We found no significant difference between the heart rate or mean arterial blood pressure responses elicited by intra-gastric administration of PBS and hydrolysate 603K or 605K or BiPRO whey protein isolate at the different doses tested.

Conclusions

The present results indicate that:
Single oral administration of hydrolysate 601 (at the doses of 75 or 150 mg/kg) in conscious unrestrained SHR significantly reduced mean arterial blood pressure from 1 to 7 hours after administration.
Blood pressure returned to the initial level at 24 hours after administration.
Conversely, the hydrolysate 603K and 605K and the control BiPRO whey protein isolate (at any of the doses tested) did not change the mean arterial blood pressure of the SHR rats in this study.

A suitable regimen for treatment with the noted hydrolysate 601 will comprise oral administration of the above doses of 75 to 150 mg/kg at intervals of from 2 to 24 hours. More broadly, the dosages and intervals could be increased or decreased by from 50 to 500 percent, as might be indicated by treatment over time.

The above description is intended to enable the person skilled in the art to practice the invention. It is not intended to detail all of the possible modifications and variations which will become apparent to the skilled worker upon reading the description. It is intended, however, that all such modifications and variations be included within the scope of the invention which is seen in the above description and otherwise defined by the following claims. The claims are meant to cover the indicated elements and steps in any arrangement or sequence which is effective to meet the objectives intended for the invention, unless the context specifically indicates the contrary.

1. A process for preparing an ACE-suppressing composition comprising:
preparing an aqueous solution of whey protein isolate and a proteolytic enzyme; holding said solution under conditions effective to partially hydrolyze said whey protein isolate to provide a hydrolysate having increased ACE-suppressing activity in mammals; and
recovering said hydrolysate from said solution.

2. A process according to claim 1 wherein the proteolytic enzyme is inactivated.

3. A process according to claim 1 wherein the proteolytic enzyme is inactivated by heating following hydrolysis.

4. A process according to claim 1 wherein the hydrolysate is dried.

5. A whey protein hydrolysate as prepared according to claim 1.

6. A treatment for a mammal to suppress ACE, said regimen comprising:
orally administering to the mammal, a product prepared according to claim 1 in amounts and at intervals effective to suppress ACE-activity.

7. A treatment regimen for a mammal to reduce symptoms of hypertension, said regimen comprising:
orally administering to the mammal, a whey protein hydrolysate prepared by treatment of a whey protein isolate as described, in amounts and at intervals effective to reduce symptoms of hypertension.

8. A treatment according to claim 7, wherein the hydrolysate is prepared from a whey protein isolate characterized as follows:

| Analysis | Specification | | Typical Range |
|---|---|---|---|
| Moisture (%) | 5.0 | max. | 4.7 ± 0.2 |
| Protein, dry basis | 95.0 | min. | 97.5 ± 1.0 |
| (N × 6.38)(%) | | | |
| Fat (%) | 1.0 | max. | 0.6 ± 0.2 |
| Ash (%) | 3.0 | max. | 1.7 ± 0.3 |
| Lactose (%) | 1.0 | max. | <0.5 |
| | | |
|---|---|---|
| pH | 6.7-7.5 | 7.0 ± 0.2 |

9. A treatment according to claim 8 wherein the enzyme employed in the hydrolysis comprises trypsin.

10. A treatment according to claim 9 wherein the enzyme employed in the hydrolysis comprises trypsin from mammal, poultry or fish sources.

11. A treatment according to claim 9 wherein the enzyme employed in the hydrolysis comprises porcine trypsin.

12. A treatment according to any of claims 7-11, wherein the hydrolysate is characterized by

| molecular weight profiles (HPLC) | |
|---|---|
| within the ranges (Daltons) | soluble peptides*** |
| >10,000 | 50-60% |
| 5,000-10,000 | 10-20% |
| 2000-5000 | 10-20% |
| <2000 | 10-20% |

Percent of total soluble peptides in 0.45 μ filtrate.

13. A treatment according to any of claims 7-12, wherein the hydrolysate is characterized by a degree of hydrolysis of from 3 to 20%.

14. A treatment according to any of claims 7-1, wherein the hydrolysate will be orally administered at a dose of 75 to 150 mg/kg at intervals of from 2 to 24 hours.
